(19) United States
(12) Patent Application Publication
(10) Pub. No.: US 2019/0142286 A1
(43) Pub. Date: May 16, 2019

(54) PHOTOPLETHYSMOGRAPHIC WEARABLE BLOOD PRESSURE MONITORING SYSTEM AND METHODS

(71) Applicant: SENSOGRAM TECHNOLOGIES, INC., Plano, TX (US)

(72) Inventor: Vahram Mouradian, Plano, TX (US)

(21) Appl. No.: 15/365,242

(22) Filed: Nov. 30, 2016

Related U.S. Application Data
(63) Continuation-in-part of application No. 14/674,499, filed on Mar. 31, 2015, now Pat. No. 9,936,885.
Application No. 14/674,499 is a continuation-in-part of application No. 14/675,639, filed on Mar. 31, 2015, now Pat. No. 10,117,586.
(60) Provisional application No. 61/972,905, filed on Mar. 31, 2014.
(60) Provisional application No. 61/973,035, filed on Mar. 31, 2014.

(51) Int. Cl. A61B 5/021; A61B 5/024; A61B 5/00; A61B 5/026

(52) U.S. Cl. CPC A61B 5/02108; A61B 5/02416; A61B 5/7275; A61B 2560/0223; A61B 5/0261; A61B 5/6826; A61B 5/7246

(57) ABSTRACT

A method for estimating blood pressure, including: identifying representative PPG pulse curve shapes associated with first and second direct non-invasive blood pressure measurements; generating at least one blood pressure correlation function representing at least a relationship between a first difference between the first shape and the second shape and a second difference between the first direct blood pressure measurement and the second direct blood pressure measurement; obtaining a measured PPG pulse signal from a patient; identifying a measured representative shape of a measured PPG pulse curve from the measured PPG pulse signal; and generating an estimated blood pressure based on the measured representative shape and the at least one blood pressure correlation function.

This application is a continuation-in-part of commonly-owned U.S. application Ser. No. 14/674,499 (“APPARATUS FOR AMBIENT NOISE CANCELLATION IN PPG SENSORS”) filed on Mar. 31, 2015, and claims priority to U.S. provisional patent application No. 61/972,905, filed on Mar. 31, 2014. This application is also a continuation-in-part of commonly-owned U.S. application Ser. No. 14/675,639 (“CONTINUOUS NON-INVASIVE WEARABLE BLOOD PRESSURE MONITORING SYSTEM”) filed on Mar. 31, 2015, and claims priority to U.S. provisional patent application No. 61/973,035, filed on Mar. 31, 2014, the disclosures of which are incorporated herein by reference for all purposes.

### TECHNICAL FIELD

The invention relates in general to photoplethysmographic (PPG) measurement systems and apparatus using optical sensors, and in particular to non-invasive human blood pressure measurements by wearable optical-sensing systems using dynamic calibration methods for periodic accuracy adjustments of such measurements.

### BACKGROUND OF THE INVENTION

Blood pressure measurement techniques are generally classified as either instant or continuous. Instant blood pressure measurement means checking blood pressure at certain points in time, like a spot check, while continuous measurement is checking a patient's blood pressure constantly, with every heartbeat. Instant techniques of blood pressure measurement involve some kind of sensor working for a short period of time, such as sphygmomanometer cuffs that operate non-invasively. The disadvantage of such techniques is that they can miss intermittent blood pressure changes, and the administration of the measurement can be cumbersome, difficult, and uncomfortable. This is particularly true where the subject is under intensive care or is in an assisted living environment (e.g., the elderly), in which blood pressure measurements are taken frequently. Continuous blood pressure measurement techniques typically employ an invasive device such as an arterial catheter, from which instant blood pressure can be tracked in real time. Continuous blood pressure measurement devices have the disadvantage of being invasive to the body, and often are not amenable to use outside controlled environments, such as the surgical theater.

Photoplethysmography or photoplethysmographic (PPG) systems have been used in an attempt to measure various physiological characteristics including, but not limited to, the blood-oxygen saturation of hemoglobin in arterial blood, the volume of individual blood pulsations supplying the tissue, and the rate of blood pulsations corresponding to each heartbeat of a patient. Attempts at measuring some of these characteristics have used a non-invasive PPG sensor, which scatters light through a portion of the patient's tissue where blood is perfused through the blood vessels (capillaries and arteries) and optically senses the absorption and/or reflection of light in such tissue.

Typical PPG measurement systems include an optical sensor worn on the tip of a patient's appendage (e.g., a finger, an earlobe, etc.). The sensor has a photoemitter that directs light signals into the appendage where the sensor is attached, and a photoreceiver that detects light reflected by or transmitted through the tissue. In the reflection mode, some portion of light is absorbed and the remaining portion is reflected back to the photoreceiver. In the transmission mode, some portion of light is absorbed, and the remaining portion is transmitted through the tissue to the photoreceiver. The operation mode depends on the configuration and intended use of the optical sensor. For example, fingertip sensors are often configured to operate in the reflection mode, whereas earlobe sensors are often configured to operate in transmission mode. The intensity of the light received by the photoreceiver is monitored to provide one or more intensity signals, which can be resolved into a waveform indicating relative values of blood flow rate at the measured location. These intensity signals are used to compute blood parameters, but the waveform produced by the signal does not directly indicate blood pressure.

There remains a need to provide alternative techniques and systems for measuring blood pressure.

### SUMMARY

In a first aspect, there is provided a method for estimating blood pressure. The method includes identifying a first representative shape of a first photoplethysmographic (“PPG”) pulse curve associated with a first direct blood pressure measurement, the first direct blood pressure measurement comprising a non-invasive measurement; identifying a second representative shape of a second PPG pulse curve associated with a second direct blood pressure measurement, the second direct blood pressure measurement being different from the first direct blood pressure measurement, the second direct blood pressure measurement comprising a non-invasive measurement; generating at least one blood pressure correlation function representing at least a relationship between a first difference between the first shape and the second shape and a second difference between the first direct blood pressure measurement and the second direct blood pressure measurement; obtaining a measured PPG pulse signal from a patient; identifying a measured representative shape of a measured PPG pulse curve from the measured PPG pulse signal; and generating an estimated blood pressure based on the measured representative shape and the at least one blood pressure correlation function.

The process of identifying the first representative shape may include identifying a first PPG data set obtained concurrently with the first direct blood pressure measurement, and evaluating the first PPG data set to identify a plurality of first user descriptive points (“UDP”), each first UDP comprising at least one of a representative amplitude and a representative time of a respective one of a plurality of predetermined PPG curve shape characteristics. The process of identifying the second representative shape may include identifying a second PPG data set obtained concurrently with the second direct blood pressure measurement; and evaluating the second PPG data set to identify a plurality of second UDPs, each second UDP comprising at least one of a representative amplitude and a representative time of a respective one of the plurality of predetermined PPG curve shape characteristics. The process of generating the at least one blood pressure correlation function may include evaluating the first UDPs and the second UDPs to identify one or more relationships between the first UDPs and the second UDPs corresponding to a difference between the first direct blood pressure measurement and the second blood pressure measurement. The process of generating the estimated blood pressure may include evaluating the measured representative shape of the measured PPG pulse curve to identify one or more measured UDPs, each measured UDP comprising at least one of a representative amplitude and a representative time of a respective one of the plurality of predetermined PPG curve shape characteristics, and applying one or more of the measured UDPs to the at least one blood pressure correlation function to generate an estimated blood pressure associated with the measured PPG pulse signal.

The method also may include identifying a third PPG data set obtained concurrently with a third direct blood pressure measurement, the third direct blood pressure measurement comprising a non-invasive measurement, and evaluating the third PPG data set to identify a plurality of third UDPs, each third UDP comprising at least one of a representative amplitude and a representative time of a respective one of the plurality of predetermined PPG curve shape characteristics. The process of generating the at least one blood pressure correlation function may include evaluating the first UDPs, the second UDPs and the third UDPs to identify one or more relationships between the first UDPs, the second UDPs and the third UDPs corresponding to a difference between the first direct blood pressure measurement, the second blood pressure measurement and the third direct blood pressure measurement.

The method also may include identifying a fourth PPG data set obtained concurrently with a fourth direct blood pressure measurement, the fourth direct blood pressure measurement comprising a non-invasive measurement, and evaluating the fourth PPG data set to identify a plurality of fourth UDPs, each fourth UDP comprising at least one of a representative amplitude and a representative time of a respective one of the plurality of predetermined PPG curve shape characteristics. The process of generating the at least one blood pressure correlation function may include evaluating the first UDPs, the second UDPs, the third UDPs and the fourth UDPs to identify one or more relationships between the first UDPs, the second UDPs, the third UDPs and the fourth UDPs corresponding to a difference between the first direct blood pressure measurement, the second blood pressure measurement, the third direct blood pressure measurement and the fourth direct blood pressure measurement.

Evaluating the first PPG data set may include identifying a plurality of PPG pulses within the first PPG data set, evaluating the PPG pulses to determine whether the PPG pulses pass one or more quality criteria, and selecting the first UDPs from one or more of the PPG pulses that pass the one or more quality criteria. The quality criteria nay include at least a first requirement that the baseline value of a selected PPG pulse to be within a predetermined range, and a second requirement that the selected PPG pulse can be resolved to identify a respective UDP for each of a minimum number of the plurality of predetermined PPG curve shape characteristics.

Each of the plurality of predetermined PPG curve shape characteristics may be a respective defined portion of a curve representing a single PPG pulse with amplitude as a function of time and a total pulse width defined as a different in time between a start point of the curve and an end point of the curve. The respective defined portions may include comprise two or more of: a first UDP representing a maximum amplitude of the curve; a second UDP representing a maximum value of a first derivative of the curve located with respect to time after the start point of the curve and before the maximum amplitude of the curve; a third UDP representing a minimum value of the first derivative of the curve located with respect to time after the maximum amplitude of the curve and before 50% of the total pulse width; a fourth UDP representing a maximum of curvature of the curve located between the third UDP and a first zero crossing of a second derivative of the curve that is within a predetermined time of the first zero crossing; a fifth UDP representing a first zero crossing of the second derivative of the curve that is located with respect to time after the fourth UDP and before 70% of the total pulse width; a sixth UDP representing a maximum of curvature of the curve between the fifth UDP and a minimum of the first derivative of the curve located with respect to time between the fifth UDP and 85% of the total pulse width, and within a predetermine time of the fifth UDP; a seventh UDP representing the minimum of the first derivative of the curve located with respect to time between the fifth UDP and 85% of the total pulse width; an eighth UDP representing a maximum of curvature of the curve located with respect to time between the seventh UDP and a maximum of the first derivative of the curve after the seventh UDP that is located within a predetermined time of the maximum of the first derivative of the curve after the seventh UDP; and a ninth UDP representing the maximum of the first derivative of the curve after the seventh UDP. The respective defined portions may include at least the first UDP, the second UDP, the fourth UDP and the sixth UDP. The respective defined portions may also include the third UDP and the fifth UDP.

Generating the at least one blood pressure correlation function may include identifying a first expression having one or more variables, and evaluating the first expression using a first group of one or more first UDPs and a corresponding first group of one or more second UDPs to generate a first correlation function correlating a difference between the first group of one or more first UDPs and the first group of one or more second UDPs to a difference between the first direct blood pressure measurement and the second direct blood pressure measurement. Evaluating the first expression may include performing at least one of a linear regression analysis or a polynomial fit analysis. The first expression may be one of: BP=f(aᵢ/aⱼ); BP=f(tᵢ/tⱼ); BP=f(tᵢ−tⱼ); BP=f [(tᵢ−tⱼ)/t₀]; and BP=f [(aᵢ/aⱼ)*(tᵢ/tⱼ)]; where BP is blood pressure, a represents an amplitude value, t represents a time value, subscript i represent a first individual UDP in the first group of one or more first UDPs, subscript i represent a second individual UDP in the first group of one or more first UDPs, and t₀ is a total time of the PPG pulse.

The method also may include evaluating the first expression using a second group of one or more first UDPs and a corresponding second group of one or more second UDPs to generate a second correlation function correlating a difference between the second group of one or more first UDPs and the second group of one or more second UDPs to a difference between the first direct blood pressure measurement and the second direct blood pressure measurement, determining a first correlation function quality score, determining a second correlation function quality score, ranking the first correlation function and the second correlation function based on the values of the first correlation function quality score and the second correlation function quality score, and selecting the highest ranked of the first correlation function and the second correlation function as the blood pressure correlation function. Evaluating the first expression may include performing at least one of a linear regression analysis or a polynomial fit analysis, and the first correlation function quality score and the second correlation function quality score each may be a respective least squares residual value or a respective r-squared value, and ranking the first correlation function and the second correlation function may include ranking based on a statistical match between the respective correlation function and the difference between the first direct blood pressure measurement and the second direct blood pressure measurement.

Generating the at least one blood pressure correlation function may include identifying a plurality of expressions having one or more variables, and evaluating each of the plurality of expressions using a respective first group of one or more first UDPs and a respective corresponding first group of one or more second UDPs to generate a respective first correlation function correlating a difference between the respective first group of one or more first UDPs and the respective first group of one or more second UDPs to a difference between the first direct blood pressure measurement and the second direct blood pressure measurement. The method also may include evaluating a quality metric of each of the respective first correlation functions, assigning a quality rank to each of the respective first correlation functions based on the respective quality metric, and selecting, as the blood pressure correlation function, a one of the respective first correlation functions having a highest quality rank. The method also may include evaluating each of the plurality of expressions using a respective second group of one or more first UDPs and a respective corresponding second group of one or more second UDPs to generate a respective second correlation function correlating a difference between the respective second group of one or more first UDPs and the respective second group of one or more second UDPs to a difference between the first direct blood pressure measurement and the second direct blood pressure measurement. The method may further include evaluating a quality metric of each of the respective first correlation functions and each of the respective second correlation functions, assigning a quality rank to each of the respective first correlation functions and each of the respective second correlation functions based on the respective quality metric, and selecting, as the blood pressure correlation function, a one of the respective first correlation functions and the respective second correlation functions having a highest quality rank. Evaluating each of the plurality of expressions may include performing at least one of a linear regression analysis or a polynomial fit analysis, and evaluating a quality metric comprises evaluating a respective least squares residual value or a respective r-squared value.

Generating the at least one blood pressure correlation function my include generating a plurality of candidate blood pressure correlation functions based on a corresponding plurality of relationships between a corresponding first difference between the first shape and the second shape and a corresponding second difference between the first direct blood pressure measurement and the second direct blood pressure measurement, ranking the plurality of candidate blood pressure correlation functions, and selecting the highest ranked candidate blood pressure correlation functions as the at least one blood pressure correlation function. Generating the plurality of candidate blood pressure correlation functions may include performing a regression analysis on values of predetermined points on the first representative shape of the first PPG pulse curve and values of predetermined points on the second representative shape of the second PPG pulse curve. Ranking the plurality of candidate blood pressure correlation functions may include evaluating a respective statistical quality of each of the plurality of candidate blood pressure correlation functions. The respective statistical quality may be at least one of a residual value and an r-squared value. Ranking the plurality of candidate blood pressure correlation functions could also include evaluating a magnitude of the corresponding difference between the first shape and the second shape for each respective candidate blood pressure correlation function, and rejecting candidate blood pressure correlation functions having a magnitude below a predetermined threshold.

In another aspect, there is provided an instrument configured for performing the method described above and other blood pressure measurement methods. The instrument may include a PPG sensor and processor configured to analyze PPG signals in conjunction with directly measured blood pressure values, and other details and features such as discussed, by way of example, herein.

### BRIEF DESCRIPTION OF THE DRAWINGS

Embodiments of the invention will now be described, strictly by way of example, with reference to the accompanying drawings, in which:

FIG. 1 is a schematic illustration of a vital signs monitoring system.

FIG. 2 illustrates an embodiment of an optical sensor subsystem.

FIG. 3 is a schematic illustration of the optical sensor subsystem of FIG. 2.

FIG. 4 is a plot of an exemplary PPG signal.

FIG. 5 is a plot of an exemplary PPG pulse signal.

FIG. 6 is a plot of an exemplary PPG pulse signal annotated to indicate exemplary user descriptive points.

FIG. 7 is a flowchart illustrating a method for providing a correlation between PPG pulse signal information and blood pressure.

FIG. 8 is a flowchart illustrating a method for identifying PPG calibration data.

FIG. 9 is an exemplary illustration of a function derived from PPG pulse data to represent the change in a patient's blood pressure.

FIG. 10 is a flowchart illustrating a method for monitoring a patient's blood pressure.

FIG. 11 is an example of a vital signs monitoring system.

### DESCRIPTION OF THE EMBODIMENTS

It has been found that PPG systems are a good candidate for both continuous and instant measurement of blood pressure, despite the fact that the PPG signal does not directly indicate blood pressure. The following disclosure provides examples of a blood pressure monitoring system that uses a non-invasive PPG device, along with a novel method and programming for monitoring blood pressure. Aspects of the present invention may be used as a wearable, non-invasive blood pressure (NIBP) monitor allowing instant, on the spot, and mobile, remote readings of blood pressure data from close proximity as well as from remote locations via the Internet or other wired or wireless communication systems.

Specific examples of components, signals, messages, protocols, and arrangements are described below to simplify the present disclosure. These are, of course, merely examples and are not intended to limit the invention from that described in the claims. Well-known elements are presented without detailed description in order to not obscure the present disclosure with unnecessary detail. For the most part, details, unnecessary to obtain a complete understanding of the present invention, have been omitted where such details are within the understanding of persons of ordinary skill in the art in the relevant industry. For example, details regarding control circuitry used to control the various elements described herein are omitted, as such control circuits are within the scope of persons of ordinary knowledge in the relevant industry. Similarly, details of PPG sensors such as the construction and operation of the photoemitter and photodetector and the physical shape and configuration of the PPG sensor are omitted as such are known in the art.

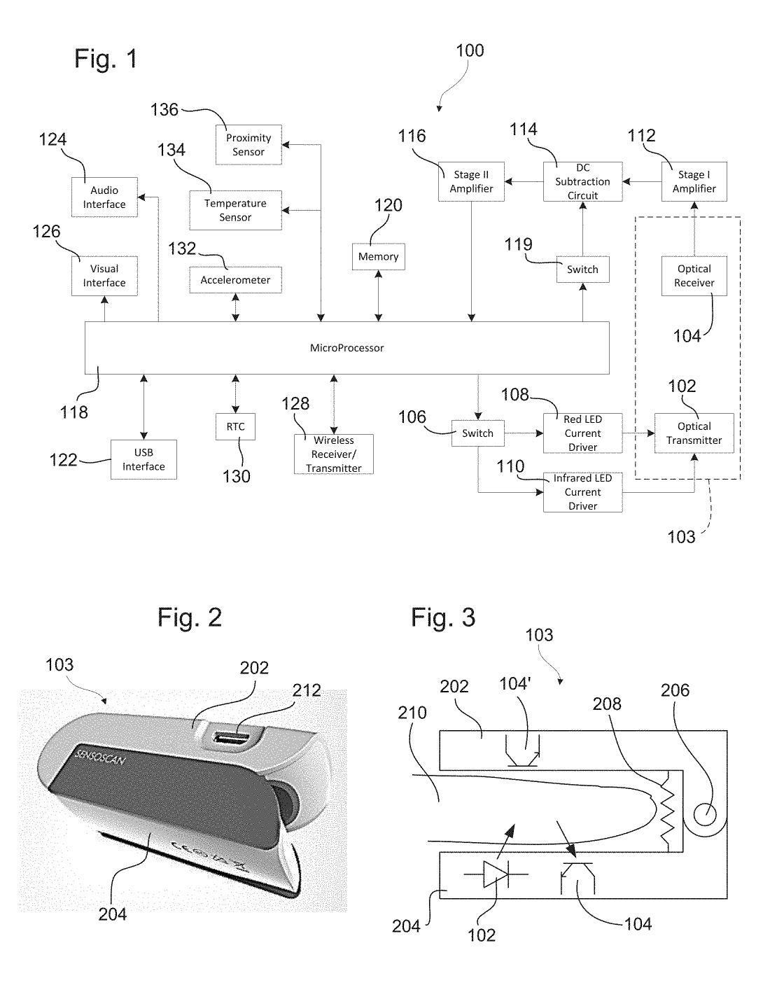

FIG. 1 illustrates a conceptual functional diagram of a vital signs monitoring system 100. The system 100 may be a self-contained operating unit (e.g., a single instrument that performs all relevant steps and outputs the desired information), a collection of operating units (e.g., parts that may operate independently and be operatively connected either in combined function or by data transfer to provide the desired information), or any combination of operating units. For ease of explanation, this specification generally describes the system 100 without differentiating between different physical or operative components, but it will be appreciated that components and functions may be allocated among a number of operatively interrelated parts.

The system 100 includes an optical sensor subsystem 103 which may be configured as a pulse oximeter system. The optical sensor subsystem 103 is designed to assist in the measurement of a user's vital signs, such as heart rate, respiration rate and oxygen saturation, by using non-invasive methods. For instance, absorption of light by oxyhemoglobin and deoxyhemoglobin are significantly different at red light and infrared light. By measuring the difference in absorbance at various wavelengths, the degree of blood oxygen saturation can be estimated, as known in the art. The present disclosure also contemplates using the optical sensor subsystem 103 to assist with detecting blood pressure, as explained in more detail below.

The optical sensor subsystem 103 is positioned on a portion of the user's tissue. For instance, the optical sensor subsystem 103 may be configured to mount on a finger (e.g., in a ring or finger clamp worn by the user), ear lobe, wrist (e.g., in a watch worn by the user), or on other parts of the body. The optical sensor subsystem 103 includes one or more optical transmitters 102, which are configured to transmit light at one or more predetermined wavelengths, and one or more optical receivers 104 that are configured to receive the transmitted light from the optical transmitter 102.

FIGS. 2 and 3 illustrate an embodiment of an optical sensor subsystem 103 that may be used with embodiments of the invention. The exemplary optical sensor subsystem 103 has a clamp-like structure having an upper arm 202 and a lower arm 204 that are joined by a hinge 206. A spring 208 biases the upper arm 202 and lower arm 204 to rotate about the hinge 206 to apply a force to retain the optical sensor subsystem 103 on the patient's body. In this example, the device is configured as a fingertip clamp that fits on a patient's finger 210, but other embodiments may be configured to fit on other appendages (e.g., earlobes or the like) or body parts. The optical sensor subsystem 103 also may include one or more wired connections (not shown), communication ports 212, wireless communication circuits, power supplies (e.g., batteries), or the like, as known in the art.

One or more optical transmitters 102, and one or more optical receivers 104, 104′ are provided in the upper arm 202 and/or lower arm 204. FIG. 3 shows two locations for optical receivers. A first optical receiver 104 is positioned on the same arm as the optical transmitter 102 for reflective mode operation. A second optical receiver 104′ is located on the arm opposite the optical transmitter 102 for transmission mode operation.

In the reflection mode configuration, the optical transmitter 102 and the optical receiver 104 are positioned adjacent to each other so that the optical receiver 104 may receive light originated by the optical transmitter 102 and reflected by the user's tissue. In other words, the optical transmitter 102 transmits light to penetrate the skin to the blood underneath, some portion of the transmitted light is absorbed by the blood in the area of reach of the penetrated light, and another portion of the light is reflected by the blood and received by the optical receiver 104.

In the transmission mode configuration, an optical receiver 104′ is positioned in opposition to the optical transmitter 102, such that the optical receiver 104′ may receive light that has passed through the blood. One portion of the emitted light will be absorbed by the blood, and another portion will pass through the blood to strike the optical receiver 104. Of course, portions of the light in either mode also may be absorbed or reflected by non-blood components of the body, but such may be minimized by selection of the light's wavelength or accounted for by other means such as background noise subtraction and the like, as known in the art.

In either mode, the optical receiver 104, 104′ generates a signal corresponding to the intensity of the light that reaches the optical receiver 104, 104′. To this end, the optical receiver 104, 104′ may comprise a photodiode or the like. Photodiodes generate a current that is proportional to the intensity of the received light—the greater the light received, the greater the current generated by the photodiode.

In actual embodiments, the optical sensor subsystem 103 may omit one of the other of the optical receivers 104, 104′ to operate exclusively in reflection or transmission mode.

The light to be transmitted through the user's tissue may be selected to be of one or more wavelengths that are absorbed by the blood in an amount representative of the amount of the blood constituent present in the blood vessel. The amount of transmitted light scattered through or reflected from the tissue will vary in accordance with the changing amount of blood constituent in the blood vessel and the related light absorption.

For example, in certain embodiments, the optical transmitter 102 may have a red light-emitting diode (“LED”) that transmits red light at a wavelength of about 580 to 660 nm, and an infrared LED that emits infrared light at a wavelength of about 880 to 940 nm. The two LEDs of the optical transmitter 102 are controlled by a LED controller circuit, which may selectively activate the two LEDs by controlling their respective current management schemes. The LED controller circuit may comprise, for example, a digital to analog converter (“DAC”), a switch 106, a red LED current driver 108, and an infrared LED current driver 110. A microprocessor 118 (which may be a single processor or any collection of cooperating processors) is connected via MCU pins to the switch 106, and is programmed to commute a control voltage from the DAC to the LEDs at appropriate intervals via the switch 106 and the LED current drivers 108 and 110. The DAC converts digital signals to an analog signal such as current or voltage. Control systems for optical transmitters 102 such as described above are generally known in the art and require no further explanation herein.

The optical receiver 104 may comprise one or more photodiodes configured to detect light at particular wavelengths. For example, one photodiode may detect light at a wavelength of about 580 to 660 nm, and a second photodiode may detect light at a wavelength of about 880 to 940 nm. Other embodiments may use a single photodiode, and the light of different wavelengths, such as red and infrared, may be time multiplexed to differentiate between light emitted from the two different LEDs. The signal detected by a single photodiode may be demultiplexed to extract the two different light signals. The demultiplexing frequency preferably is high enough so that it is much larger than the blood pulse rate, to provide sufficient resolution for accurately evaluating the received signal. Such devices and their operation and control are known in the art and need not be described further herein.

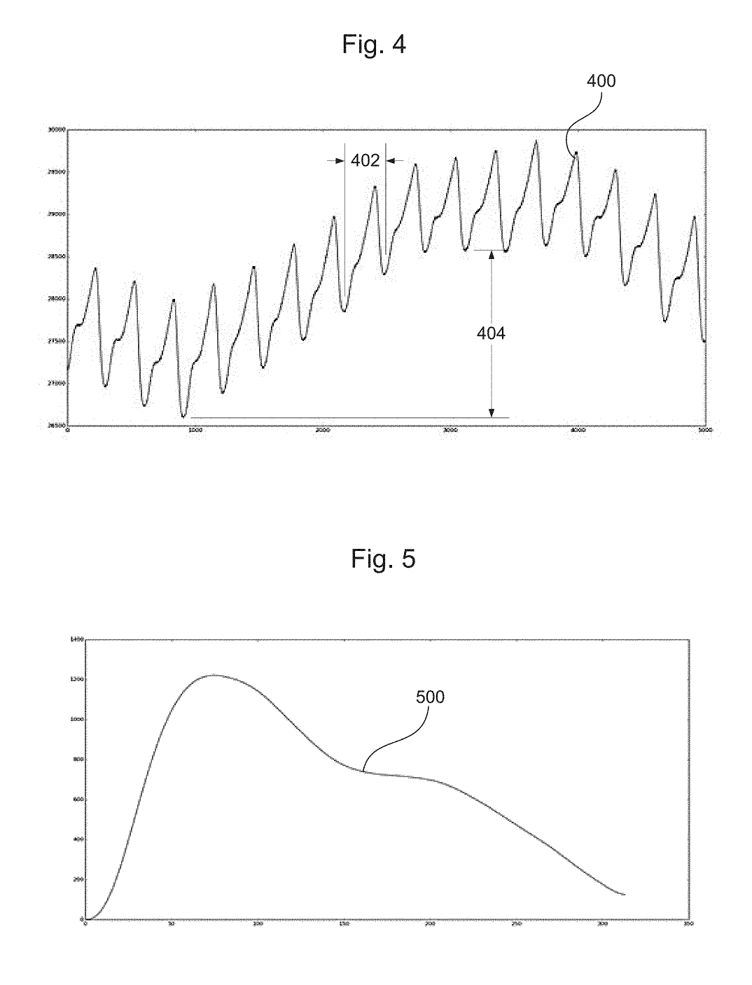

The current generated by the optical receiver 104 may be sent to a first stage amplifier 112, such as a first stage trans-impedance amplifier, to amplify the received current. The amplified current or output signal from the first stage amplifier 112 typically comprises a DC component and an AC component. The AC component represents the periodic change of light received by the optical receiver 104, which is a function of the change in the volume or other characteristics of the blood within the patient's blood vessels and tissue. The DC component represents ambient physiological and system-generated noise present in the received light. The blood change in the vessel can be visualized in the form of an AC component accompanied by the noise represented in the form of the DC component (see, e.g., FIGS. 4 and 5).

A DC subtraction circuit 114 may be provided as an offset input to a second stage operational amplifier 116. The DC subtraction circuit 114 extracts the DC component of the signal, so that only the AC portion of the signal is amplified by the second stage amplifier 116.

An analog-to-digital (“A/D”) converter (not shown) receives the amplified current from the second stage operational amplifier 116, and converts it into a digital waveform which is then sent to a microprocessor 118.

In certain embodiments, the system 100 also may include an ambient noise reduction circuit, which reduces noise due to ambient light effect in the received current using a switch 119. Details of a suitable ambient noise reduction circuit are found in the jointly owned and co-pending patent application “Apparatus for Ambient Noise Cancellation in PPG Sensors,” application Ser. No. 14/674,499, filed on Mar. 31, 2015, the specification of which is incorporated by reference into this application.

The microprocessor 118 receives the digital waveform from the second stage amplifier 116 via the A/D converter as an input signal. Any suitable microprocessor may be used, but an ultra-low power microprocessor is preferred. In one embodiment, the microprocessor 118 or “MCU” may be based on the 32 bit ARM Cortex-M4 core, which includes a variety of peripheral devices. The microprocessor may have an ultra-low power consumption of about 238 pA/MHz in dynamic run mode, and 0.35 pA in lowest power mode. Although low energy, the core of the microprocessor 118 is powerful enough to allow collection and processing of data from the sensors on the fly.

The microprocessor 118 is coupled to at least one memory 120 for storing post-processed data and for firmware instructions. In certain embodiments, the memory 120 may be approximately 256 Mb of serial flash memory. The microprocessor 118 also may be in communication with a USB (“Universal Serial Bus”) interface 122, such as a micro USB interface. The USB interface 122 may be coupled to a USB-to-UART (“Universal Asynchronous Receiver and Transmitter”) converter (not shown), such as an enhanced UART with a USB interface. Among other features, the USB interface 122 allows the microprocessor 118 to communicate with an external computing device via a wired USB cable. In certain embodiments, the USB interface 122 also supports USB suspend, resume and remote wakeup operations. Program instructions residing in the memory 120 may be updated via the USB interface 122 (e.g., firmware) and where necessary data may be transferred between the microprocessor 118 and the computing device.

The USB interface 122 is also maybe coupled to the system's power supply circuit (not shown) and can receive direct current to charge and recharge a portable power supply, such as a lithium-ion polymer battery (not shown). In some embodiments, the power supply may be coupled to an on/off controller which is also coupled to an on/off switch. In certain embodiments, the system's power supply can be inductively charged. As such power supply circuits are well known in the industry, the power supply circuit will not be discussed in detail in this disclosure.

In the shown embodiment, the microprocessor 118 sends control signals to the optical transmitter 102, which begins transmitting light to be received by the optical receiver 104 to start the data gathering process. The microprocessor 118 also performs data acquisition and analysis on the received digital waveforms from the second stage amplifier 116.

Data from the optical receiver 104, which is referred to herein generally as the “PPG signal” may be stored in the memory 120 for later transmission or use. The microprocessor 118 may store the PPG signal in a raw form (e.g., as received from the optical receiver 104 without amplification and/or DC subtraction), partially processed (e.g., amplified but with the DC component included), fully processed into a digital waveform, as pure computed results (e.g., as a calculated pulse rate or the like without retaining the raw data from which the calculation is made), or in other forms, as desired. Data stored in the memory 120 and calculated results of the patient's vital signs may also be sent to a number of user interface devices. For example, in certain embodiments, the calculated results may be sent to an audio interface 124 such as an earphone speaker. In other embodiments, the calculated results may be sent to a visual interface 126, such as an LCD display or touch sensitive screen via a display driver (not shown).

Additionally, the raw data and calculated results may also be sent to a wireless transceiver 128. The wireless transceiver 128 may be any suitable device, such as a near-field communication device, a Bluetooth radio capable of communication with a smart phone or other such Bluetooth-capable computing devices, and so on. In certain embodiments, the Bluetooth radio may be a low energy “system on a chip” or “SOC.” The SOC may include a microcontroller core with Flash memory and static RAM. In certain embodiments, the SOC also includes a Bluetooth v4.0 low energy front-end. In certain embodiments, the SOC may be used as a Network processor, which provides Bluetooth Low Energy connectivity. In other embodiments, a ZIGBEE protocol or any other point-to-point wireless protocol (standard or non-standard) may be incorporated into the wireless transceiver. Other alternatives will be apparent to persons of ordinary skill in the art in view of the present disclosure.

The microprocessor 118 may also be connected to a number of other system components and peripherals. For example, the microprocessor 118 may be connected to a Real Time Clock (“RTC”) 130, an accelerometer/gyroscope 132, a temperature sensor 134, and/or a proximity sensor 136. Examples of such devices are described below.

The RTC 130 may be a RTC module with a built-in crystal oscillating at 32.768 kHz, 1 MHz Fast-mode Plus (Fm+) with a two wire I2C interface. Such an RTC module may have a wide interface operating voltage: 1.6-5.5 V, Wide clock operating voltage: 1.2-5.5 V and ultra-low power consumption: 130 nA typ @ 3.0V/25° C.

The accelerometer/gyroscope 132 may be an intelligent, low-power, 3/6/9-axis accelerometer/gyroscope with 12 bits of resolution. In certain embodiments, the accelerometer may be provided with embedded functions with flexible user-programmable options, configurable to two interrupt pins. For instance, embedded interrupt functions enable overall power savings, by relieving the host processor from continuously polling data. There may be access to either low-pass or high-pass filtered data, which minimizes the data analysis required for jolt detection and faster transitions. In certain embodiments, the accelerometer 132 may be configured to generate inertial wake-up interrupt signals from any combination of the configurable embedded functions, enabling the accelerometer/gyroscope 132 to monitor inertial events while remaining in a low-power mode during periods of inactivity.

The temperature sensor 134 may be a digital output temperature sensor in a four-ball wafer chip-scale package (WCSP) capable of reading temperatures to resolution of 1° C. In certain embodiments, the temperature sensor 134 has a two-wire interface that is compatible with both I2C and SMBus interfaces. In addition, the interface supports multiple devices on the bus simultaneously, eliminating the need to send individual commands to each temperature sensor on the bus. In certain embodiments, the voltage requirements vary between 1.4V to 3.6V.

The proximity sensor 136 allows the presence of a nearby object to be detected. The proximity sensor 136 may be a self-contained, self-calibrating digital IC which projects a touch or proximity field to several centimeters through any dielectric. Certain embodiments may be coupled to a capacitor for operation. The proximity sensor 136 may be useful to suspend operation when the system is not in proximity to a user, or to indicate if the system has been removed. Other uses will be apparent in view of the present disclosure.

The system 100 includes a program application to process the PPG signal to estimate certain physical conditions of the user. For instance, the program application may be configured to evaluate the PPG signal to determine the patient's pulse, blood oxygen saturation, respiration rate, and other vital signs or physiological properties. The program application may be stored in the memory 120 and executed by the microprocessor 118, but other storage and processing systems may be used.

Blood flow is pulsatile in nature, and this causes the amount of transmitted or reflected light received by the optical receiver 104 to change with time. The PPG signal generated by the optical receiver 104 (which usually is measured as a change in voltage or current over time) reflects this pulsatile nature, as shown in the exemplary PPG signal plot in FIG. 4. Here, the PPG signal 400 is plotted with amplitude on the y axis and time on the x axis. The PPG signal includes a number of distinct pulses 402 caused by individual heartbeats, and the overall amplitude of the pulses 402 can vary through a noticeable range 404 as a result of respiration, muscular contraction, repositioning of the body, and so on. This data can be processed in various ways to provide information such as respiratory rate (e.g., by wavelet transform techniques, etc.), and so on. In conventional systems, this PPG signal 400 may be analyzed to determine the patient's heart rate by converting each peak into a square wave and averaging the time between square waves, or by other algorithms as known in the art.

FIG. 5 illustrates a portion of the PPG signal 400 after the signal has been filtered and inverted, and the DC component has been removed to leave only the AC component. The resulting portion of the PPG signal 400 comprises a waveform 500 of a single heartbeat pulse, again with amplitude on the y axis and time on the x axis. Waveforms of a single heartbeat pulse such as this are referred to herein as a “PPG pulse.” Conventional techniques can be used to perform this filtering, inverting and DC subtraction, as known in the art.

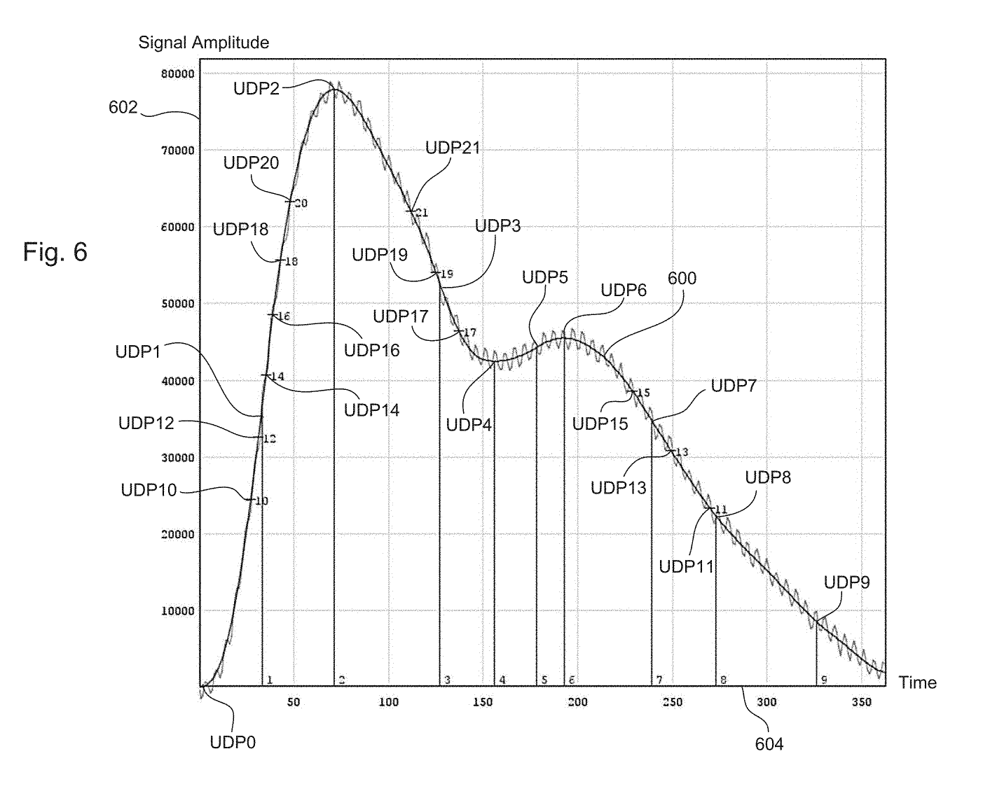

FIG. 6 illustrates an exemplary PPG pulse 600 annotated to indicate various points of potential interest, as explained in more detail below. The PPG pulse 600 is shown after being subjected to mathematical and numerical transformation and conditioning processes (e.g., amplification, filtering, removal of the DC component, analog-to-digital conversion, normalization, etc.) to improve accuracy and clarity. Such processes may be performed by the processor 118 or other circuitry, and are generally known in the art and need not be described herein in detail. The PPG pulse 600 in FIG. 6 illustrates the signal amplitude (e.g., measured current or voltage) on the y axis 602 plotted against time on the x axis 604. The signal amplitude is, of course, proportional to and representative of the patient's blood flow rate.

A PPG pulse 600 can provide a variety of information about a patient's physical condition, but each individual's blood flow properties (and thus, the patient's measured PPG signal and PPG pulse) can react differently to changes in blood pressure. Still further, each individual can having uniquely-shaped PPG pulses, and the individual's PPG pulses can change in shape depending on the location at which the PPG signal is measured. Thus, the PPG pulse 600 itself does not specifically indicate the individual's blood pressure in an absolute sense. While PPG systems are in common use to provide information such as pulse rate, and blood oximetry, these convenient and non-invasive devices have not been conventionally used to provide blood pressure measurements. For blood pressure, most patients instead must endure the use of invasive blood pressure monitoring devices, or the discomfort and inconvenience of repeated administration of blood pressure measurement using sphygmomanometers and the like.

It has now been discovered, however, that PPG pulse data can be accurately correlated to a patient's blood pressure, and used to provide continuous or instantaneous blood pressure monitoring in a comfortable and convenient manner. In general terms, the invention works by correlating, at an individual level, changes in PPG signals to changes in blood pressure, and using those correlations to evaluate future blood pressure levels. An exemplary process for establishing this correlation is described with reference to FIGS. 6 through 8.

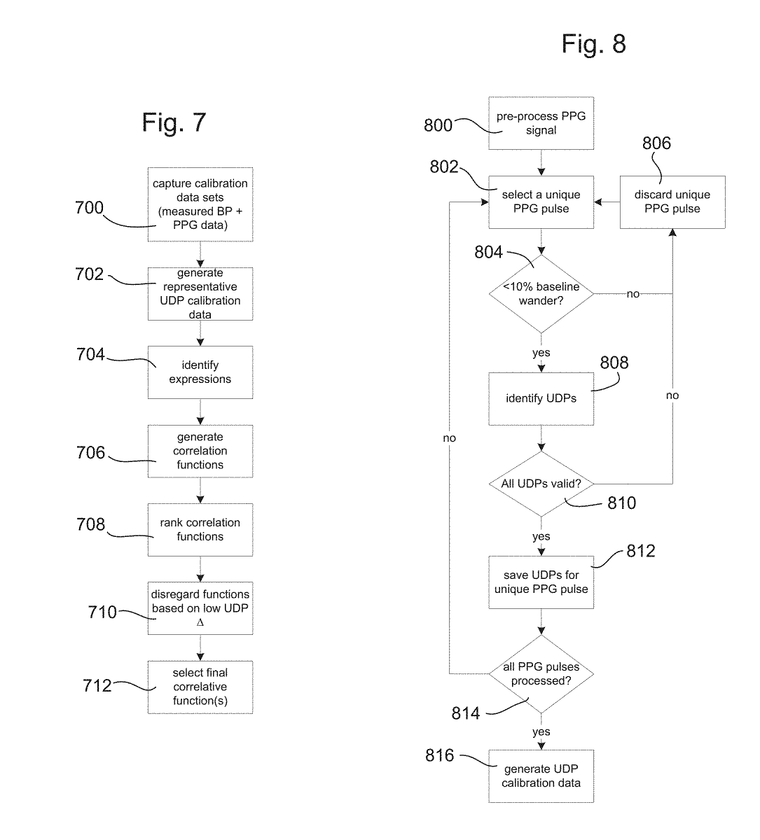

Referring specifically to FIG. 7, the process for correlating PPG signals to blood pressure begins at step 700 by capturing a number of calibration data sets from the individual patient. This can be accomplished by directly measuring the patient's blood pressure using conventional means such as a sphygmomanometer, catheter or the like, while concurrently using a PPG device such as the optical sensor subsystem 103 described above, to detect the patient's PPG signal. The blood pressure measurement taken during the capture of calibration data sets is referred to as the direct blood pressure measurement. The PPG signal captured during the capture of calibration data sets is referred to as the PPG data or PPG data set. The direct blood pressure measurement and PPG data preferably are obtained concurrently, meaning that they are taken at approximately same time or within a time window when the patient's blood pressure is not expected to change significantly. It is not required for the PPG data set to be obtained during the actual direct blood pressure measurement process, and if it is expected that the process of taking the direct blood pressure measurement could affect the PPG data, the patient may be allowed a short time interval between the direct blood pressure measurement and the collection of the PPG data. As another example, the PPG data collection may be started immediately after the diastolic pressure is read using a conventional sphygmomanometer blood pressure measurement process. As still another example, the direct blood pressure measurement may be taken shortly or immediately after the PPG data is collected, or as the PPG data collection comes to an end. These and other timings would all be considered concurrent measurement to obtain a PPG data set that correlates to the direct blood pressure measurement, provided no overt change in the patient's physical condition occurs between obtaining the direct blood pressure measurement and PPG data (e.g., changing from sitting to standing, gross motor movements, etc.).

Step 700 involves the capture of multiple calibration data sets so as to provide representative PPG data for different values of the direct blood pressure measurement. In one example, four separate calibration data sets are obtained using a sphygmomanometer cuff on the patient's arm and a PPG device such as described above. Two calibration data sets are obtained with the patient's cuffed arm at heart level, a third calibration data set is obtained the cuffed arm above the patient's head (to obtain a relatively low blood pressure), and a fourth calibration data set is obtained with the patient's arm at the patient's side (to obtain a relatively high blood pressure).

Each calibration data set may be collected, for example, by taking a single direct blood pressure measurement (systolic/diastolic) with the sphygmomanometer cuff, while also (preferably simultaneously) collecting a PPG data set comprising 30 seconds of PPG data acquisition at a sampling rate of 512 Hz. In other embodiments, each calibration data set may include multiple averaged direct blood pressure measurements, other PPG data acquisition durations or rates, and so on. It will also be readily apparent that the methods and systems described herein can be used with any type of direct blood pressure measurement that may be used as an alternative to a sphygmomanometer.

In step 702, each calibration data set is analyzed to generate a set of representative user descriptive points (“UDPs”). The representative UDPs are an indication of one or more typical PPG curve shape characteristics (slope, magnitude, differential time, etc.) of the patient's PPG pulse for the direct blood pressure measurement value obtained with the calibration data set. The representative UDPs may be generated by evaluating the individual PPG pulse for each measured heartbeat in the PPG data. For example, each PPG pulse can be analyzed to identify a number of particular distinct UDPs that are expected to be in each PPG pulse, and then averaging the values of the measured UDPs for each individual PPG pulse within the PPG data to provide the representative UDPs for the particular direct blood pressure measurement taken at the same time as the PPG data. An example of this process is now described with reference to FIGS. 6 and 8.

The exemplary process for generating the representative UDPs begins at step 800. In step 800, the PPG data set is pre-processed by performing one or more mathematical and numerical transformation and conditioning processes (e.g., amplification, filtering, removal of the DC component, analog-to-digital conversion, normalization, etc.) to improve accuracy and clarity of the PPG signal. Such processes are well-known in the art and may be performed using the system illustrated in FIG. 1 or a comparable system, and the details of the pre-processing methodologies need not be described herein. Portions of the PPG data set also may be cropped to exclude portions of the data (e.g., removing 10 seconds of data at the beginning to account for artifacts caused by the direct blood pressure measurement, patient anxiety or discomfort, or the like), or otherwise manipulated.

Portions of step 800 may be performed at different times. For example, a first stage of amplification may be performed before the PPG signal data is saved to a memory as the PPG data. The signal processing measures in step 800 may be performed in a lossless manner (e.g., retaining a record of the original unaltered PPG signal), a semi-lossless manner (e.g., retaining some filtered or extracted data, such as keeping a record of DC magnitude values removed from the signal), and so on. It will also be appreciated that steps that might be considered pre-processing steps also may be delayed to various times after the other process steps, such as by removing the DC component after step 804 described below. Pre-processing also may be performed on an as-needed basis, such that the pre-processing steps necessary to support a subsequent operation are delayed until immediately or shortly before those subsequent operations are performed.

After the PPG signal is pre-processed in step 800 (or as part of the pre-processing), the PPG signal is evaluated to identify unique PPG pulses for individual heartbeats that occurred during the calibration data set collection process. As noted above, each PPG pulse represents a single heartbeat. The PPG data can include any number of unique PPG pulses, but it is preferred for the PPG data to include at least ten useable PPG pulses, which may require collecting well over ten heartbeats of data. To this end, the PPG data may be collected for a time period that is expected to provide the desired number of useable PPG pulses (e.g. 30 seconds), and if an insufficient number of PPG pulses are provided the process can return to step 700 to re-collect the calibration data sets. As another example, the step 702 of generating representative UDPs may be performed during the calibration data set collection step 700, so that the process of steps 700 and 702 only end once a suitable number of PPG pulses have been collected (or if there is a manual interrupt). Other alternatives will be apparent to persons of ordinary skill in the art in view of the present disclosure.

At step 802, a first unique PPG pulse is selected for further processing. At step 804, the selected PPG pulse is evaluated to determine whether the PPG pulse has an excessive amount of baseline wander. Baseline wander is a measurement of any difference between the starting and ending value of the PPG pulse on the y axis 602. Such differences can be caused by a significant transition in the patient's blood pressure during the course of the PPG data collection process, which can be caused by a sudden movement or the like. Artifacts caused by these circumstances can adversely affect the calibration process, and preferably are excluded. In the present example, the baseline wander is measured by comparing the magnitude of the signal at the beginning of the PPG pulse with the magnitude of the signal at the end of the PPG pulse. If the difference in magnitude exceeds a predetermined threshold, the PPG pulse is rejected. For example, the magnitudes of the PPG pulse can be measured with both values including their DC component. If the difference in the two values is less than 10%, the PPG pulse is processed further in step 808. If the difference in the two values is greater than 10%, the PPG pulse is discarded and the process moves back to step 802 to select a new unique PPG pulse for processing. In this example, step 804 may be performed before the DC component is removed by pre-processing, by performing step 804 after removing the DC component by pre-processing but referring to stored values of the DC component values for the PPG pulse, or by other techniques that will be apparent in view of this disclosure.

In step 808, the PPG pulse 600 is processed to identify the UDPs. Any suitable number of UDPs may be identified for each PPG pulse. In the example illustrated in FIG. 6, twenty-two UDPs are identified by reference numbers UDP0 to UDP21. To support the UDP selection, noise reduction and curve-fitting may be performed to reduce or eliminate local data variations along the curve. Each PPG pulse 600 may be stored as a function or in tables or the like, as desired and as necessary to accommodate any computing requirements or limitations. FIG. 6 shows the smoothed PPG pulse 600 laid over the unsmoothed data. A filter such as a 16 Hz, 6^{th} order Butterworth low-pass filter may be used to assist with this process, but other filters and smoothing techniques may alternatively be used.

FIG. 6 illustrates the following UDPs, with x being defined as the PPG curve as a function of time, x′ being the first derivative thereof, and x″ being the second derivative thereof:
UDP0: First sample of x.
UDP1: Maximum of x′ between UDP0 and UDP2.
UDP2: Maximum of x.
UDP3: Minimum of x′ between UDP2 and before 50% of the PPG pulse width.
UDP4: Maximum of curvature between UDP3 and UDP5 that is within 50 samples of UDP5 (at a sampling rate of 512 Hz this corresponds to about 0.098 seconds).
UDP5: First zero crossing of x″ after UDP4 and before 70% of the PPG pulse width.
UDP6: Maximum of curvature between UDP5 and UDP7 that is within 50 samples of UDP5.
UDP7: Minimum of x′ after UDP5 and before 85% of the PPG pulse width.
UDP8: Maximum of curvature between UDP7 and UDP9 that is within 30 samples of point 9.
UDP9: Maximum of x′ after UDP7.
UDP10: first point where x rises above 30% of maximum value.
UDP11: First point where x drops below 30% of maximum value.
UDP12: First point where x rises above 40% of maximum value.
UDP13: First point where x drops below 40% of maximum value.
UDP14: First point where x rises above 50% of maximum value.
UDP15: First point where x drops below 50% of maximum value.
UDP16: First point where x rises above 60% of maximum value.
UDP17: First point where x drops below 60% of maximum value.
UDP18: First point where x rises above 70% of maximum value.
UDP19: First point where x drops below 70% of maximum value.
UDP20: First point where x rises above 80% of maximum value.
UDP21: First point where x drops below 80% of maximum value.

The UDPs may be determined using first and second derivative values of the PPG pulse, curvature of the signal, relative vector analysis, pulse decomposition techniques, and other methods known in the art. The PPG pulse width may be determined as the total time or number of samples along the x axis 604 between UDP0 and a subsequent “foot” in the PPG signal indicating a local low point in the blood flow through the tissue. The identification of individual PPG pulses and their pulse widths from a PPG signal is known in the art and need not be explained further herein.

The foregoing UDPs are selected for various reasons. For example, UDP2 represents the maximum pressure point, UDP3 indicates the closing of the aorta valve, UDP4 indicates the local minimum pressure before the reflecting wave arrives, and UDP6 indicates the reflecting wave pressure. UDP7-UDP9 can indicate a secondary wave and identify the capillary vessels' and venules' response to the same. UDP10-UDP21 allow the calculation of certain time intervals between different pulse amplitude levels (e.g., 30%-80% at 10% increments). Other UDPs may be selected, and the foregoing UDPs may be omitted or replaced in other embodiments.

Each UDP indicates a particular value and location of the signal on the PPG pulse, as plotted by relative amplitude versus relative time. The amplitude and time may be converted to an arbitrary scale (e.g., 0.00-1.00). The absolute amplitude and time values preferably are not used, which makes the methodologies described herein independent of the PPG signal's AC and DC levels. This can help eliminate potential inaccuracies that may be caused by variables such as the amplitude of the PPG signal being affected by phenomena other than the patient's blood pressure.

In step 810, the collected UDP data is evaluated to ensure that the UDPs are valid. Validity can be determined using any number of error-checking algorithms based on the expected physiological behavior of the patient. For example, values that are above or below a certain threshold (e.g., an amplitude reading of zero for UDP4, or negative amplitude readings for any point) may be considered invalid. Values that are out of the expected order (e.g., UDP2 being before UDP1, or UDP5 and UDP6 being at the same location on the x axis 604) also may be considered invalid.

If any UDP is found to be invalid, the process returns to step 806 to discard the PPG pulse and proceed with selecting a new PPG pulse. Alternatively, the process may allow some threshold error level before deciding to return to step 806. For example, apparent errors in UDP10 through UDP21 may lead to omitting those UDPs from later calculations, but retaining the remainder of the PPG pulse for purposes of other subsequent calculations. As another alternative, certain UDPs may be deemed critical (e.g., UDP1-UDP7) and the PPG pulse will only be discarded if these are somehow invalid. Other alternatives will be apparent to persons of ordinary skill in the art in view of the present disclosure.

If the UDPs are found to be valid (or if some minimum required selection of UDPs are found to be valid), the process saves the UDPs for the unique PPG pulse in step 812, and proceeds to step 814 to determine whether there are any remaining PPG pulses to evaluate. If there are more PPG pulses to evaluate, the process returns to step 802. If not, the process proceeds to step 816.

If desired, the process may include a minimum successful PPG pulse count before moving to step 816. For example, the process may require the PPG data set to include at least ten valid PPG curves (i.e., curves that are processed to step 812 to identify their UDPs), and if this number is not satisfied the system 100 may instruct the operator to return to step 700 to collect additional calibration data sets. Also, the process may proceed to step 816 if a sufficient number of PPG pulses are processed to step 812, even if there are more unprocessed PPG pulses. Other alternatives will be apparent to persons of ordinary skill in the art in view of the present disclosure.

At step 816, the process generates representative UDP calibration data for the direct blood pressure measurement taken in step 700. The representative UDP calibration data may comprise, for example, a computation of the arithmetic mean average value of each UDP (both amplitude and time) in the PPG pulses saved in step 812. These values are saved as a representative set of UDP values (i.e., UDP0-UDP21). These representative UDP values may be stored in the memory 120 as arrays or tables, curve-fitted, represented by equations or functions, and so on. In other embodiments, more complex averaging and statistical analysis may be performed on the data to arrive at the representative UDP values, and the representative amplitude for each UDP value may be determined using different averaging techniques than the representative time for each UDP value. Other alternatives may be used in other embodiments.

Referring back to FIG. 7, the process of generating representative UDP calibration data 702 (such as described in the process of FIG. 8) is repeated for each calibration data set. At the end of step 702, each direct blood pressure measurement will be associated with representative UDP calibration data for that blood pressure. For example, for an individual patient, representative UDP calibration data may be established for direct blood pressure measurements of 120/80, 125/86, 118/75 and 115/72. The direct blood pressure measurements and representative UDP calibration data are stored in the memory 120.

After defining the representative UDP calibration data, the process moves on to correlating the representative UDP calibration data to the direct blood pressure measurement values to identify representative relationships between changes in blood pressure and changes in the patient's UDP data. It has been found that this correlation can be different for different individuals. For example, in some individuals, the ratio of UDP2 to UDP6 can be proportional or otherwise correlated to changes in blood pressure, whereas in other individuals there may be no discernable correlation between changes in UDP2 and UDP6 and changes in the individual's blood pressure. To account for these differences, the system evaluates a number of different relationship expressions to determine which, if any, provide a correlation between blood pressure and UDP data.

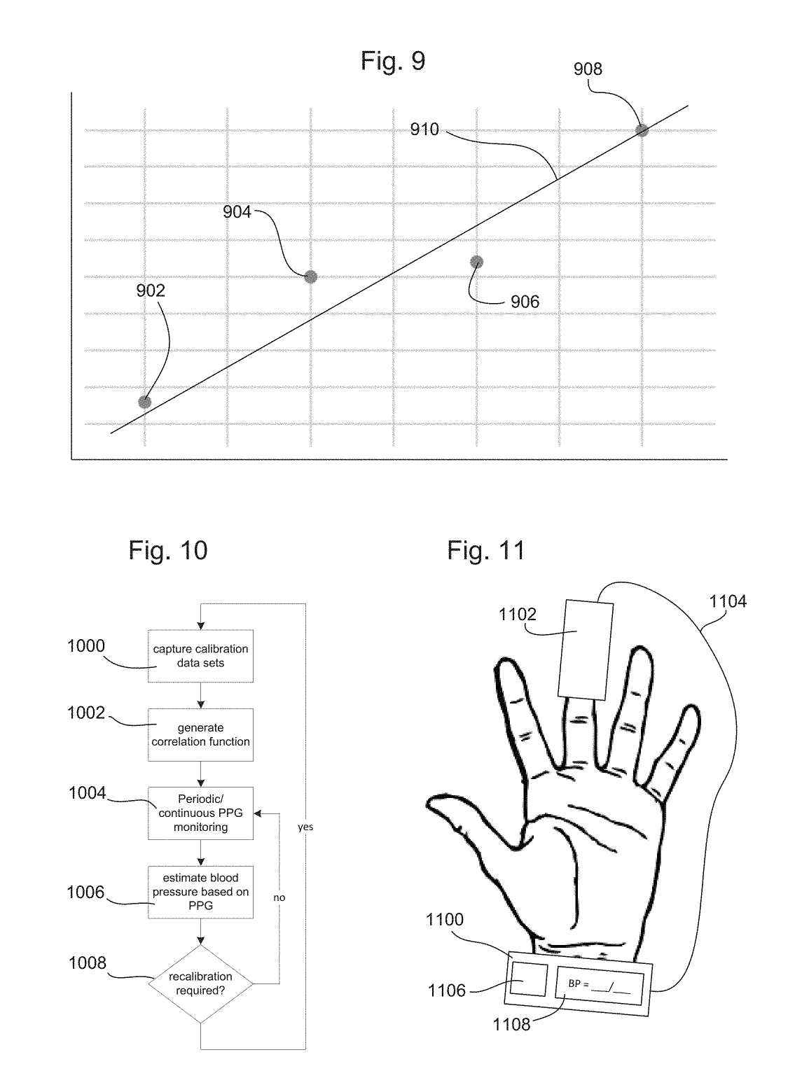

The expressions comprise a set of one or more predefined mathematical hypotheses. Each expression may be evaluated in relation to one or more UDPs to find a relationship between changes in the UDPs and changes in the direct blood pressure measurements. For example, a first expression may be BP=f(aᵢ/aⱼ), where BP is blood pressure, aᵢ is the amplitude (y axis) value of a first representative UDP (e.g., UDP2), and aⱼ is the amplitude of a second representative UDP (e.g., UDP6) on the same representative UDP curve. Using this exemplary expression, the system evaluates the ratio aᵢ/aⱼ for the corresponding representative UDPs related to each of the direct blood pressure measurements (e.g., evaluate the ratio UDP2/UDP6 for each direct blood pressure measurement) to see whether the patient's blood pressure is a discernable function (f) of the ratio. In a simple case, for example, the ratio of UDP2/UDP6 may increase linearly in a manner that approximates a linear increase in the patient's direct blood pressure measurements (e.g., a change of 5% in blood pressure correlates to a 5% change in UDP2/UDP6). This example is illustrated in FIG. 9, in which the four direct blood pressure measurements are identified as points 902, 904, 906 and 908, and the value of UDP2/UDP6 mathematically fits a linear function 910 that increases proportionally to the increase in direct blood pressure measurement. In this case, the function f can be resolved into a simple first degree polynomial (i.e., linear) equation.

In practice, the function may be more complicated than a simple linear relationship, and the value for the function f may ultimately be a polynomial equation of any degree or the like. The system can use any number of curve-fitting, polynomial regression, vector analysis, interpolation or function-fitting algorithms or the like to evaluate whether a function can be solved to correlate the expression to the direct blood pressure measurement data.

Referring back to FIG. 7, in step 704 the system selects one or more expressions. The list of expressions preferably is the same in every case, but the system may select among different expressions if certain expressions are not deemed suitable for the underling PPG data or direct blood pressure measurements. For example, where the patient has a PPG pulse profile that categorically excludes a feature, such as the absence of a minimum before the reflected wave (i.e., a lack of UDP4), expressions that are designed to test whether this feature is a candidate to represent the patient's blood pressure may be excluded.

Exemplary expressions that may be selected in step 704 include: BP=f(aᵢ/aⱼ); BP=f(tᵢ/tⱼ); BP=f(tᵢ−tⱼ); BP=f [(tᵢ−tⱼ)/t₀]; and BP=f [(aᵢ/aⱼ)*(tᵢ/tⱼ)], where BP is blood pressure, a represents an amplitude value (y axis value), t represents a time value (x axis value), subscripts i and j represent first and second representative UDPs on the representative PPG pulse, and t₀ is the total width of the PPG pulse. The foregoing expressions resolve the blood pressure into a function of the amplitudes and/or times of one or more UDPs. Other expressions may be used, as desired, and alternative expressions may compare properties of a curve fitted to the representative UDPs (as opposed to UDP point values). For example an expression may relate the blood pressure as a function of the area under all or a portion of a curve-fitted representation of the representative UDPs (e.g., the area under the curve between UDP1 and UDP3). Other alternatives will be apparent to persons of ordinary skill in the art in view of the present disclosure.

In step 706, the selected expressions are evaluated to generate one or more candidate correlation functions providing a relationship between changes in UDP values and changes in direct blood pressure measurement. Each expression may be evaluated for each possible combination of UDPs (e.g., the expression BP=f(aᵢ/aⱼ) may be separately evaluated for all possible combinations of UDP0-UDP21). While computationally intensive, this process may identify unexpected and reliable correlations between the PPG data and the patient's direct blood pressure measurement. Moreover, computational resources can be quite powerful and may be able to perform such processing without undue delay, making the scope of this task inconsequential. In other cases, however, certain expressions can be limited to being evaluated with only a limited set of the UDPs. Such limitations may be based on physiological insights. For example, where there is not expected to be any physiological reason for a particular UDP (or a relationship between multiple UDPs in the same PPG pulse) to correlate with blood pressure, such UDPs can be excluded from one or more expressions. For example, the ratio aᵢ/aⱼ, where aᵢ is the amplitude of UDP11 and aⱼ is the amplitude of UDP10, is expected to be approximately 1 under all circumstances and evaluation of this ratio can be skipped.

In other cases, the expression may reflect certain specific insights about possible relationships between physiology and blood pressure. For example, it is believed that the velocity of the reflected pulse wave in the body may increase with blood pressure due to the expectation that more highly pressurized liquids are relatively dense, and will convey pressure waves more quickly. Therefore the expression BP=f [(tᵢ−tⱼ)/t₀] may be selected and used with the time value of UDP6 as tᵢ, and the time value of UDP2 as tⱼ, to evaluate whether the time gap between the peak pulse and the reflected pulse wave changes in a manner that correlates with changes in the patient's direct blood pressure measurements (dividing by t₀ in this expression normalizes the time difference to the scale of the pulse). The same fundamental expression also can be used with alternative UDPs that might correlate a change in velocity of the reflected pulse wave to a change in blood pressure (e.g., comparing change in time between UDP1 and UDP6). Other alternatives will be apparent to persons of ordinary skill in the art in view of the present disclosure.

The exact expressions and the UDPs that are to be evaluated with those expressions to attempt to find a suitable correlation function with the patient's direct blood pressure measurements may predetermined. For example, the system may be programmed to evaluate each of the four expressions discussed above on every possible combination of UDPs, or one or more expressions may be evaluated only with particular UDPs. The choice of expressions and corresponding UDPs also may be made dynamically by the system. For example, the system may include a default set expressions and corresponding UDPs to use with those expressions, but these may be changed depending on the outcome of the process of generating representative UDP calibration data 702 to account for peculiarities of the patient's PPG data. For example, a patient's PPG pulses may typically lack a distinct dicrotic notch (“saddle”) at UDP4, in which case the system may dynamically choose not to evaluate certain expressions based on a comparison of the amplitude or time position of UDP4 with other UDP values because such comparisons may not provide reliable results. Other variations will be appreciated by those skilled in the art with practice of the invention disclosed herein.

In an exemplary embodiment, the system is programmed to evaluate the following combinations of expressions and UDPs:

To identify normalized time difference correlations, the expression BP=f [(tᵢ−tⱼ)/t₀] is evaluated to provide candidate correlation functions based on the following combinations of tᵢ and tⱼ: t_{UDP17} and t_{UDP16} (i.e., BP=f [(t_{UDP17}−t_{UDP16})/t₀]; t_{UDP15} and t_{UDP14}; t_{UDP11} and t_{UDP2}; t_{UDP19} and t_{UDP18}; t_{UDP8} and t_{UDP2}; t_{UDP7} and t_{UDP2}; t_{UDP11} and t_{UDP10}; and, t_{UDP13} and t_{UDP12}.

To identify absolute time difference correlations, the expression BP=f (tᵢ−tⱼ) is evaluated to provide candidate correlation functions based on the following combinations of tᵢ and tⱼ, respectively: t_{UDP7} and t_{UDP2}; t_{UDP8} and t_{UDP2}; t_{UDP9} and t_{UDP2}; and t_{UDP22} and t_{UDP2}.

To identify time ratio correlations, the expression BP=f (tᵢ/tⱼ) is evaluated to provide candidate correlation functions based on the following combinations of tᵢ and tⱼ, respectively: t_{UDP1} and t_{UDP0}; t_{UDP2} and t_{UDP0}; and, t_{UDP3} and t_{UDP2}.

To identify amplitude correlations, the expression BP=f (aᵢ/aⱼ) is evaluated to provide candidate correlation functions based on the following combinations of aᵢ and aⱼ, respectively: a_{UDP1} and a_{UDP2}; a_{UDP3} and a_{UDP2}; a_{UDP3} and a_{UDP1}; a_{UDP4} and a_{UDP1}; a_{UDP5} and a_{UDP1}; a_{UDP2} and a_{UDP6}; a_{UDP2} and a_{UDP5}; and, a_{UDP17} and a_{UDP2}.

To identify first derivative-based correlations, the expression BP=f (aᵢ/dⱼ) (where d is the value of the first derivative at UDP j) is evaluated to provide candidate correlation functions based on the following combinations of aᵢ and dⱼ, respectively: a_{UDP17} and d_{UDP17}; and, a_{UDP17} and d_{UDP16}. Additional first-derivative based correlations are evaluated using the expression and variables BP=f [(d_{UDP1}−d_{UDP3})/a_{UDP2}].

To identify amplitude and time ratio based correlations, the expression BP=f [(aᵢ/aⱼ)*(tᵢ/tⱼ)] is evaluated to provide candidate correlation functions based on the following combinations of aᵢ, aⱼ, tᵢ, tⱼ, respectively: a_{UDP1}, a_{UDP2}, t_{UDP1} and t_{UDP2}; a_{UDP2}, a_{UDP3}, t_{UDP2} and t_{UDP3}; a_{UDP2}, a_{UDP4}, t_{UDP2} and t_{UDP4}; a_{UDP1}, a_{UDP4}, t_{UDP1} and t_{UDP4}; a_{UDP1}, a_{UDP5}, t_{UDP1} and t_{UDP5}; a_{UDP2}, a_{UDP5}, t_{UDP2} and t_{UDP5}; a_{UDP3}, a_{UDP4}, t_{UDP3} and t_{UDP4}; a_{UDP3}, a_{UDP5}, t_{UDP3} and t_{UDP5}; a_{UDP4}, a_{UDP5}, t_{UDP4} and t_{UDP5}; a_{UDP1}, a_{UDP2}, t_{UDP3} and t_{UDP2}; and a_{UDP1}, a_{UDP5}, t_{UDP3} and t_{UDP5}.

Other combinations of expression and UDP values may be used in other embodiments.

In step 706, each selected expression is evaluated with each UDP or a subset of UDPs, to identify a correlation function to correlate changes in one or more UDPs with changes in the patient's direct blood pressure measurements. As noted above, this process can use conventional curve-fitting, vector analysis, or other algorithms to identify candidate functional relationships between the direct blood pressure measurement data and the changes in UDP values. For example, in one embodiment, a linear regression analysis is performed to compare changes in each expression to changes in direct blood pressure measurements. FIG. 9 shows an example of a plot of a linear regression analysis for one of the expressions being performed for one UDP selection (e.g., the expression BP=f(aᵢ/aⱼ) being performed with aᵢ as the amplitude of UDP2 and aⱼ as the amplitude of UDP6). The linear regression may use any suitable technique to perform the analysis, such as least squares, least absolute deviations, ridge regression, Bayesian linear regression, and so on. The linear regression provides a correlation function f plotted as line 920.

After step 706 is complete, the system may have a number of candidate correlation functions, each of which provides some possible functional relationship between changes in particular UDPs and changes in direct blood pressure measurements.

In step 708, each correlation function identified for each expression in step 706 is evaluated according to one or more metrics to determine the merits of the correlation function. Conventional mathematical models can be used to evaluate the merits of the functions. For example, where a linear regression analysis is used as explained above, the regression error or regression residuals associated with the regression may be evaluated to determine the statistical quality of the correlation function to represent the direct blood pressure measurements. The correlation functions are ranked according to the degree of match (e.g., ranked by lowest (best match) to highest (worst match) value of residuals when using a least squares analysis), such that functions that exhibit a poor match with the direct blood pressure measurements are ranked lower than those that exhibit a better match. Step 708 also may summarily exclude correlation functions that have a quality below a predetermined threshold (e.g., residuals above a certain value), or those that rank below a certain threshold relative to the other correlation functions (e.g., remove all but the three ranked highest for quality). Other alternatives and statistical quality analysis methods will be apparent to persons of ordinary skill in the art in view of the present disclosure.

It has been found that in some cases an expression can be resolved to identify a correlation function that closely correlates particular changes in UDP properties to changes in direct blood pressure measurements, but the underlying UDP values actually demonstrate very little absolute sensitivity to changes in blood pressure. For example a UDP value may change very slightly among the calibration data sets, but still appear to accurately track changes in blood pressure. In such cases, there may be a concern that the changes in the UDP value are not statistically reliable enough to support the correlation. Thus, in step 710 (which may be part of step 708), the correlation functions generated from the expressions are evaluated to eliminate those that are based on minimal changes in UDP values across differential blood pressures, regardless of how well the correlation functions generated from the expressions correlate the UDP with the changes in blood pressure. The degree of change in UDP values can be evaluating using any conventional mathematical model, such as by calculating the difference between each UDP value (x) and the population mean (μ) and dividing this by the population mean value, then averaging these values to arrive at an average percentage variation of the UDP data points. The formula Average(|x−μ|/μ) may be used for this purpose, but other methods may alternatively be used. If the value of this calculation is below a predetermined threshold (e.g., 5% or 10%), the correlation function generated by the expression is discarded or given a reduced ranking.

In step 710, functions that compare changes in signal amplitude (i.e., the y axis value) may be evaluated only to see how much the y axis values of the UDPs vary for the different direct blood pressure measurements, and changes in UDP timing (i.e., location on the x axis) may be ignored. Similarly, functions that rely on changes in UDP timing may be evaluated to see how much the x axis values of the UDPs vary for the different direct blood pressure measurements, while changes in signal amplitude may be ignored. In other cases, changes in both the signal amplitude and the UDP timing may be evaluated to ensure sufficient changes in those values to support reliance on a correlation function based on both characteristics of the PPG pulse.

In step 712, the functions remaining after steps 708 and 710 are evaluated to identify the one or ones having the best correlation to changes in direct blood pressure measurement. The selection of the best correlation may be based on the ranking in step 708 or on other mathematical models or criteria. For example, the system may begin with a regression analysis in step 708, and conclude with a weighted parameter analysis in step 712 to make the final decision of the best correlation functions or functions.

A weighted parameter analysis may be performed using any desired criteria. In one example, the weighted parameter analysis is performed by evaluating each of the top correlation functions identified in step 708 based on the underlying representative UDP values from the PPG data. For example, as noted above, the representative UDPs that are used by the expressions to develop the correlation functions may be based on average values of multiple UDP measurements. Those UDP measurements are likely to have some distribution of values, such as a Gaussian distribution of values with an observable standard deviation. During step 712, the system may evaluate the Gaussian distributions of the measured UDP values to provide another weighting factor to make the final selection of the best correlation function. For example, correlation functions with similar residual values may be sorted with preference being given to the correlation function that is based on UDP data having the lowest standard deviation, based on the following formula: FR=f[A(r²)+B(σ)], where FR is the final rank value for each correlation function, A is a first weighting variable, r² is the R-squared statistic of the correlation function, B is a second weighting variable, and a is the standard deviation value of the underlying UDP data used to generate the representative UDP (or UDPs) that are used in the correlation function. The weighting variables A and B can be selected as desired to provide the desired comparative weight for the values of r² and σ. After generating the final rank for each correlation function, the system selects the final correlation function as the one with the highest rank. Other alternatives will be apparent to persons of ordinary skill in the art in view of the present disclosure.

Finally, if the final selected correlated function does not already provide one, a best fit equation may be generated based on this function to match blood pressure against the UDP variable(s) that exhibit the correlating behavior. Alternatively, the function may be converted into an interpolated lookup table of blood pressures correlating to particular values of the UDP or relationships between multiple UDPs that form the basis of the formula.

It will be appreciated that the foregoing process is useful to identify a customized correlation between one or more UDP values and the particular individual patient's blood pressure. The process can be effectuated using conventional equipment, and automated to provide one or more correlation functions without user intervention. Once established, this correlation can be conveniently used to estimate the patient's blood pressure using nothing more than a standard PPG device (e.g., the optical sensor subsystem 130 and an associated computer processor). The correlation can be based on a single UDP value, or a comparison of UDP values (e.g., changes in time between two UDP points). The correlations can also be based on multiple separate correlation functions. For example, where the process returns three different and correlations between UDP data and blood pressure that all have approximately the same accuracy, the system may use a blended average of these correlations to estimate blood pressure. Other alternatives will be apparent to persons of ordinary skill in the art in view of the present disclosure.

A method for collecting and using the patient-specific correlative blood pressure information is illustrated in FIG. 10. At step 1000, the calibration data sets are captured as described above, by using a direct blood pressure measurement instrument and a PPG device. At step 1002, the system generates the correlation function(s) between the patient's direct blood pressure measurements and one or more characteristics of the patient's PPG signals, such as described above.

In step 1004, the patient is periodically or continuously monitored using a PPG device, to gather a PPG signal from the patient. In step 1006, the PPG signal is evaluated to estimate the patient's blood pressure. This process may be performed by conditioning the patient's PPG signal (e.g., filtering, inverting, amplifying, removing the DC component, etc.) to generate PPG pulses, and evaluating the PPG pulses to identify values for the UDPs that are used in the correlation function. Once the UDP value or values are identified, they are applied to the correlation function to provide the estimated blood pressure. To account for noise or periodic variations caused by external factors, the UDP values that are entered into the correlation function may be an average value of the UDP values across a number of PPG pulses (e.g., averaging UDP values within a rolling window of the last ten suitable PPG pulses). The system also may exclude UDP values that are determined to be spurious (e.g., outside a predetermined range), and it may perform quality checks such as those described above in relation to steps 804 and 810 to ensure that the PPG pulse signal is sufficiently defined and regular to be suitable to predict the patient's blood pressure. Other alternatives will be apparent to persons of ordinary skill in the art in view of the present disclosure. The blood pressure value generated by the correlation function is then displayed, saved or otherwise processed to assist with monitoring the condition of the patient.

The process also may include a recalibration checkpoint 1008, at which the system determines whether it may be necessary or desirable to recalibrate the system. For example, recalibration may be desirable after a predetermined period (e.g., one day), after predetermined events (e.g., following surgery), or if there is any reason to believe that the correlation function is not accurate. For example, if the correlation function comprises an average of three different correlation functions to estimate blood pressure, and one of the three functions starts giving significantly different values than the other two, it may indicate a change in the patient's condition that requires recalibration. When recalibration is required, the process returns to step 1000. Otherwise, it continues to loop through steps 1004 and 1006.

The instruments and processes described herein may be incorporated into any suitable operative configurations for use. The system preferably is completely non-invasive. For example, the optical sensor subsystem 103 may be a conventional commercially available device, and the microprocessor may be part of a desktop, laptop or tablet computer. The computer may include an input device (e.g., keyboard, mouse or the like) with which the user can enter the direct blood pressure measurement values taken in step 700. In use, the user (e.g., a nurse or doctor), attaches the optical sensor subsystem 103 to the patient's body, initiates PPG data collection, takes the patient's pulse manually, and enters the pulse measurement into the computer. The computer may be programmed to prompt data entry, instruct the user and patient on how the system operates, indicate errors (e.g., lack of or defective PPG data or an erroneous blood pressure value input), and so on, to preferably provide an intuitive and interactive process. After (or during) PPG data collection, the computer generates one or more correlation functions specifically tailored to the individual patient. The computer also may generate a record to identify which UDP values are being used to estimate the patient's blood pressure, so that the user or a physician can assess whether the such UDPs are or remain appropriate based on the patient's condition. The user also may be prompted to select among different possible suitable correlation functions to use for the patient.

The same computer system may be used to perform all of the process steps described herein, or the processes may be distributed among different computers. For example, one computer may be used to perform the processes of FIGS. 7 and 8 (including obtaining the direct blood pressure measurements), and a separate computer may be used to perform the monitoring and blood pressure estimating steps in FIG. 10. Where multiple computers are involved, the computers may communicate any necessary data (e.g., a patient profile file) via any suitable communication means (e.g., wireless, internet, direct wired, portable storage media, etc.).

In one exemplary embodiment, shown in FIG. 11, the system may comprise a single computer system in the form of a portable computer device 1100 that is operatively connected (via wire 1102 or wirelessly) to a PPG device 1104. The patient can wear the PPG device 1104 and the portable computer device 1100, such as with the PPG device 1104 on the patient's finger, and the portable computer device 1100 on the patient's arm. Calibration may be performed by manually entering direct blood pressure measurement values into the portable computer device, either directly by an input interface 1106 (touchscreen, buttons, etc.), or via a separate input such as a remote computer that communicates wirelessly with the portable computer device 1100. Calibration preferably can be done without removing the system from the patient. The portable computer device 1100 may include a display 1108 to provide information such as blood pressure, or it may send such information to a remote device for remote display. Other alternatives will be apparent to persons of ordinary skill in the art in view of the present disclosure.

It will be appreciated that the foregoing examples can provide a system and method for performing both continuous and instant blood pressure estimation. The system is non-invasive, accurate, and is not dependent upon constant calibration with a reference device before every measurement. The system also can be packaged in a wearable format, and can be relatively affordable, accurate, reliable and easy to use. Systems according to embodiments can replace a number of blood pressure monitoring devices available today, which generally are either instant- or continuous-read devices, but not both. Other advantages and uses will become apparent with study of this disclosure and practice of embodiments of the invention.

The present disclosure describes a number of new, useful and nonobvious features and/or combinations of features that may be used alone or together. The embodiments described herein are all exemplary, and are not intended to limit the scope of the inventions. It will be appreciated that the features shown and described in documents incorporated herein by reference may be added to embodiments in a manner corresponding to the use of such features in the incorporated references. It will also be appreciated that the inventions described herein can be modified and adapted in various ways, and all such modifications and adaptations are intended to be included in the scope of this disclosure and the appended claims.

1. A method for estimating blood pressure, the method comprising:
identifying a first representative shape of a first photoplethysmographic (“PPG”) pulse curve associated with a first direct blood pressure measurement, the first direct blood pressure measurement comprising a non-invasive measurement;
identifying a second representative shape of a second PPG pulse curve associated with a second direct blood pressure measurement, the second direct blood pressure measurement being different from the first direct blood pressure measurement, the second direct blood pressure measurement comprising a non-invasive measurement;
generating at least one blood pressure correlation function representing at least a relationship between a first difference between the first shape and the second shape and a second difference between the first direct blood pressure measurement and the second direct blood pressure measurement;
obtaining a measured PPG pulse signal from a patient;
identifying a measured representative shape of a measured PPG pulse curve from the measured PPG pulse signal; and
generating an estimated blood pressure based on the measured representative shape and the at least one blood pressure correlation function.

2. The method of claim 1, wherein:
identifying the first representative shape comprises:
identifying a first PPG data set obtained concurrently with the first direct blood pressure measurement, and
evaluating the first PPG data set to identify a plurality of first user descriptive points (“UDP”), each first UDP comprising at least one of a representative amplitude and a representative time of a respective one of a plurality of predetermined PPG curve shape characteristics;
identifying the second representative shape comprises:
identifying a second PPG data set obtained concurrently with the second direct blood pressure measurement, and
evaluating the second PPG data set to identify a plurality of second UDPs, each second UDP comprising at least one of a representative amplitude and a representative time of a respective one of the plurality of predetermined PPG curve shape characteristics;
generating the at least one blood pressure correlation function comprises evaluating the first UDPs and the second UDPs to identify one or more relationships between the first UDPs and the second UDPs corresponding to a difference between the first direct blood pressure measurement and the second blood pressure measurement; and
generating the estimated blood pressure comprises:
evaluating the measured representative shape of the measured PPG pulse curve to identify one or more measured UDPs, each measured UDP comprising at least one of a representative amplitude and a representative time of a respective one of the plurality of predetermined PPG curve shape characteristics, and
applying one or more of the measured UDPs to the at least one blood pressure correlation function to generate an estimated blood pressure associated with the measured PPG pulse signal.

3. The method of claim 2, further comprising:
identifying a third PPG data set obtained concurrently with a third direct blood pressure measurement, the third direct blood pressure measurement comprising a non-invasive measurement;
evaluating the third PPG data set to identify a plurality of third UDPs, each third UDP comprising at least one of a representative amplitude and a representative time of a respective one of the plurality of predetermined PPG curve shape characteristics; and
wherein generating the at least one blood pressure correlation function comprises:
evaluating the first UDPs, the second UDPs and the third UDPs to identify one or more relationships between the first UDPs, the second UDPs and the third UDPs corresponding to a difference between the first direct blood pressure measurement, the second blood pressure measurement and the third direct blood pressure measurement.

4. The method of claim 3, further comprising:
identifying a fourth PPG data set obtained concurrently with a fourth direct blood pressure measurement, the fourth direct blood pressure measurement comprising a non-invasive measurement;
evaluating the fourth PPG data set to identify a plurality of fourth UDPs, each fourth UDP comprising at least one of a representative amplitude and a representative time of a respective one of the plurality of predetermined PPG curve shape characteristics; and
wherein generating the at least one blood pressure correlation function comprises:
evaluating the first UDPs, the second UDPs, the third UDPs and the fourth UDPs to identify one or more relationships between the first UDPs, the second UDPs, the third UDPs and the fourth UDPs corresponding to a difference between the first direct blood pressure measurement, the second blood pressure measurement, the third direct blood pressure measurement and the fourth direct blood pressure measurement.

5. The method of claim 2, wherein evaluating the first PPG data set comprises:
identifying a plurality of PPG pulses within the first PPG data set;
evaluating the PPG pulses to determine whether the PPG pulses pass one or more quality criteria; and
selecting the first UDPs from one or more of the PPG pulses that pass the one or more quality criteria.

6. The method of claim 5, wherein the one or more quality criteria comprise at least:
a first requirement that the baseline value of a selected PPG pulse to be within a predetermined range; and
a second requirement that the selected PPG pulse can be resolved to identify a respective UDP for each of a minimum number of the plurality of predetermined PPG curve shape characteristics.

7. The method of claim 2, wherein each of the plurality of predetermined PPG curve shape characteristics comprises a respective defined portion of a curve representing a single PPG pulse with amplitude as a function of time and a total pulse width defined as a different in time between a start point of the curve and an end point of the curve, and wherein the respective defined portions comprise two or more of:
a first UDP representing a maximum amplitude of the curve;
a second UDP representing a maximum value of a first derivative of the curve located with respect to time after the start point of the curve and before the maximum amplitude of the curve;
a third UDP representing a minimum value of the first derivative of the curve located with respect to time after the maximum amplitude of the curve and before 50% of the total pulse width;
a fourth UDP representing a maximum of curvature of the curve located between the third UDP and a first zero crossing of a second derivative of the curve that is within a predetermined time of the first zero crossing;
a fifth UDP representing a first zero crossing of the second derivative of the curve that is located with respect to time after the fourth UDP and before 70% of the total pulse width;
a sixth UDP representing a maximum of curvature of the curve between the fifth UDP and a minimum of the first derivative of the curve located with respect to time between the fifth UDP and 85% of the total pulse width, and within a predetermine time of the fifth UDP;
a seventh UDP representing the minimum of the first derivative of the curve located with respect to time between the fifth UDP and 85% of the total pulse width;
an eighth UDP representing a maximum of curvature of the curve located with respect to time between the seventh UDP and a maximum of the first derivative of the curve after the seventh UDP that is located within a predetermined time of the maximum of the first derivative of the curve after the seventh UDP; and
a ninth UDP representing the maximum of the first derivative of the curve after the seventh UDP.

8. The method of claim 7, wherein the respective defined portions comprise at least the first UDP, the second UDP, the fourth UDP and the sixth UDP.

9. The method of claim 8, wherein the respective defined portions further comprise the third UDP and the fifth UDP.

10. The method of claim 2, wherein generating the at least one blood pressure correlation function comprises:
identifying a first expression having one or more variables; and
evaluating the first expression using a first group of one or more first UDPs and a corresponding first group of one or more second UDPs to generate a first correlation function correlating a difference between the first group of one or more first UDPs and the first group of one or more second UDPs to a difference between the first direct blood pressure measurement and the second direct blood pressure measurement.

11. The method of claim 10, wherein evaluating the first expression comprises performing at least one of a linear regression analysis or a polynomial fit analysis.

12. The method of claim 10, wherein the first expression comprises one of:
BP=f(aᵢ/aⱼ);
BP=f(tᵢ/tⱼ);
BP=f(tᵢ−tⱼ);
BP=f [(tᵢ−tⱼ)/t₀]; and
BP=f [(aᵢ/aⱼ)*(tᵢ/tⱼ)];
wherein BP is blood pressure, a represents an amplitude value, t represents a time value, subscript i represents a first individual UDP in the first group of one or more first UDPs, subscript j represents a second individual UDP in the first group of one or more first UDPs, and t₀ is a total time of the PPG pulse.

13. The method of claim 10, further comprising:
evaluating the first expression using a second group of one or more first UDPs and a corresponding second group of one or more second UDPs to generate a second correlation function correlating a difference between the second group of one or more first UDPs and the second group of one or more second UDPs to a difference between the first direct blood pressure measurement and the second direct blood pressure measurement;
determining a first correlation function quality score;
determining a second correlation function quality score;
ranking the first correlation function and the second correlation function based on the values of the first correlation function quality score and the second correlation function quality score; and
selecting the highest ranked of the first correlation function and the second correlation function as the blood pressure correlation function.

14. The method of claim 13, wherein:
evaluating the first expression comprises performing at least one of a linear regression analysis or a polynomial fit analysis;
the first correlation function quality score and the second correlation function quality score each comprises a respective least squares residual value or a respective r-squared value; and
ranking the first correlation function and the second correlation function comprises ranking based on a statistical match between the respective correlation function and the difference between the first direct blood pressure measurement and the second direct blood pressure measurement.

15. The method of claim 2, wherein generating the at least one blood pressure correlation function comprises:
identifying a plurality of expressions having one or more variables; and
evaluating each of the plurality of expressions using a respective first group of one or more first UDPs and a respective corresponding first group of one or more second UDPs to generate a respective first correlation function correlating a difference between the respective first group of one or more first UDPs and the respective first group of one or more second UDPs to a difference between the first direct blood pressure measurement and the second direct blood pressure measurement.

16. The method of claim 15, further comprising:
evaluating a quality metric of each of the respective first correlation functions;
assigning a quality rank to each of the respective first correlation functions based on the respective quality metric; and
selecting, as the blood pressure correlation function, a one of the respective first correlation functions having a highest quality rank.

17. The method of claim 15, further comprising:
evaluating each of the plurality of expressions using a respective second group of one or more first UDPs and a respective corresponding second group of one or more second UDPs to generate a respective second correlation function correlating a difference between the respective second group of one or more first UDPs and the respective second group of one or more second UDPs to a difference between the first direct blood pressure measurement and the second direct blood pressure measurement.

18. The method of claim 17, further comprising:
evaluating a quality metric of each of the respective first correlation functions and each of the respective second correlation functions;
assigning a quality rank to each of the respective first correlation functions and each of the respective second correlation functions based on the respective quality metric; and
selecting, as the blood pressure correlation function, a one of the respective first correlation functions and the respective second correlation functions having a highest quality rank.

19. The method of claim 18, wherein:
evaluating each of the plurality of expressions comprises performing at least one of a linear regression analysis or a polynomial fit analysis; and
evaluating a quality metric comprises evaluating a respective least squares residual value or a respective r-squared value.

20. The method of claim 1, wherein generating the at least one blood pressure correlation function comprises:
generating a plurality of candidate blood pressure correlation functions based on a corresponding plurality of relationships between a corresponding first difference between the first shape and the second shape and a corresponding second difference between the first direct blood pressure measurement and the second direct blood pressure measurement;
ranking the plurality of candidate blood pressure correlation functions; and
selecting the highest ranked candidate blood pressure correlation functions as the at least one blood pressure correlation function.

21. The method of claim 20, wherein generating the plurality of candidate blood pressure correlation functions comprises performing a regression analysis on values of predetermined points on the first representative shape of the first PPG pulse curve and values of predetermined points on the second representative shape of the second PPG pulse curve.

22. The method of claim 21, wherein ranking the plurality of candidate blood pressure correlation functions comprises evaluating a respective statistical quality of each of the plurality of candidate blood pressure correlation functions.

23. The method of claim 22, wherein the respective statistical quality comprises at least one of a residual value and an r-squared value.

24. The method of claim 20, wherein ranking the plurality of candidate blood pressure correlation functions comprises:
evaluating a magnitude of the corresponding difference between the first shape and the second shape for each respective candidate blood pressure correlation function; and
rejecting candidate blood pressure correlation functions having a magnitude below a predetermined threshold.
